(19) United States
(12) Patent Application Publication
(10) Pub. No.: US 2015/0351916 A1
(43) Pub. Date: Dec. 10, 2015

(54) SYSTEM AND METHOD FOR THREE DIMENSIONAL PRINTED IMPLANTATION GUIDES

(71) Applicants: FRANK J. KOSAREK, Charlotte, NC (US); Michelle J. Rivera, Charlotte, NC (US); Frank J. Kosarek, Charlotte, NC (US)

(72) Inventors: FRANK J. KOSAREK, Charlotte, NC (US); Michelle J. Rivera, Charlotte, NC (US); Frank J. Kosarek, Charlotte, NC (US)

(21) Appl. No.: 14/646,263

(22) PCT Filed: Feb. 4, 2015
(86) PCT No.: PCT/US15/14374

§ 371 Date: May 20, 2015

Related U.S. Application Data
(60) Provisional application No. 61/995,749, filed on Apr. 21, 2014.
(60) Provisional application No. 62/123,084, filed on Nov. 7, 2014.

(51) Int. Cl. A61F 2/30; A61B 17/17; A61B 8/08; A61B 10/02; A61B 5/055; A61B 6/03; A61B 17/88; A61F 2/46

(52) U.S. Cl. CPC A61F 2/30942; A61B 17/8808; A61B 17/17; A61F 2/4603; A61B 10/025; A61B 5/055; A61B 6/032; A61B 8/0875; A61F 2/4657; A61B 2017/00526

(57) ABSTRACT

A process for fabricating size-specific, customized bio-printed musculoskeletal tissue using three dimensional data collected from radiologic imaging is provided. Also, provided is a guide that is created from radiological imaging that demarcates the area of surgical interest. The guide is 3D printed according to guide dimensions collected from radiological imaging, including, but not limited to, CT imaging scans, CT arthrography, ultrasound, MRI, MR arthrography, or any other imaging modality used to image the musculoskeletal system.

### FIELD OF THE INVENTION

The present invention relates generally to a process for fabricating size-specific, customized bio-printed musculoskeletal tissue using three dimensional data collected from radiologic imaging. The present invention also relates to a guide that is created from radiological imaging that demarcates the area of surgical interest. The guide is 3D printed according to guide dimensions collected from radiological imaging, including, but not limited to, CT imaging scans, CT arthrography, ultrasound, MRI, MR arthrography, or any other imaging modality used to image the musculoskeletal system.

### BACKGROUND OF THE INVENTION

Bioprinting is a novel science which produces the automated fabrication of human tissue and organs using a three-dimensional (“3D”) bioprinter. In this field, tissues are created by using living cells as tiny building blocks and printing these blocks along with matrix on to sheets of biopaper. Rather than the more antiquated system of building tissue using prefabricated polymeric or decellularized tissue scaffolding to hold or draw cells into place, highly cellular tissues are generated in bioprinting by the precise placement of cells and matrix simultaneously on sheets of biopaper. These sheets of cells and matrix are stacked one upon the other in order to fabricate a 3D organ or tissue.

While great strides have been made in this area, a reproducible process that may be used to fabricate size-specific, customized bio-printed musculoskeletal tissue for use in various orthopedic applications is lacking. Specifically, a method to create precisely sized, shaped and contoured osteochondral and chondral graft material to be placed in defects on the articular surface of joints has not been developed. In addition, no precise method or guide to aid in the exact placement of these osteochondral or chondral grafts on joint surfaces exists. Precise placement using a guide is needed to ensure that the portion of the articular surface operated on is in the precise location of the diseased articular cartilage surface. Known technologies do not address this need.

Presently, there are two major types of osteochondral grafts, ALLOgraft and AUTOgraft. ALLOgraft can be fresh cadaveric osteochondral ALLOgraft material or fresh frozen osteochondral ALLOgraft material. Both of these osteochondral ALLOgraft materials are problematic for several reasons.

Fresh cadaveric ALLOgraft material is difficult to obtain from a donor patient as it has to be acquired and implanted in a short period of time from the deceased donor to the recipient. This leads to logistic problems related to speedy harvest and delivery of the fresh cadaveric ALLOgraft material. To address this problem, fresh frozen ALLOgraft cadaveric material has been used, however, it is problematic because it has been frozen, the cartilage and osseous cell viability within the tissue is decreased.

Further, both frozen and fresh osteochondral ALLOgraft material lack exact sizing capabilities, which vary in non-biologically identical patients. An area harvested from the same area in a donor's femoral condyle does not exactly match that of the recipient in many cases. Because of this incongruity, the donor osteochondral graft is usually modified by the orthopedic surgeon in the operating room to fit better into the recipient site, which is not very precise and can be a rather lengthy undertaking. Moreover, ALLOgraft is problematic because of possible immunogenic response from the recipient against the implanted osteochondral tissue. Finally, problems with cross-infection from the donor material to the recipient from numerous diseases including HIV, Hepatitis B have been reported.

Osteochondral AUTO graft material may also be harvested from the patient's native tissue in the same affected joint or from a different joint. This is problematic for several reasons. Firstly, the amount of tissue removed which is transferred to another diseased portion of the joint is small because one does not want to significantly compromise the structural integrity of an unaffected portion of the joint. Secondly the tissue and cell lines harvested are not expanded in vitro, thus limiting the amount of tissue to be implanted into the affected portion of the joint. Thirdly, the chondral tissue harvested within the same joint or another joint is difficult to match exactly in size, shape and contour to the area of the joint to be replaced. Finally, harvesting osteochondral tissue from another joint is possible, however requires additional surgery and morbidity.

Therefore, a reproducible process that fabricates size-specific, customized bio-printed musculoskeletal tissue for use in various orthopedic applications, specifically replacement of chondral and osteochondral defects in joint with size specific osteochondral and chondral graft material, is needed. Also needed is a 3-D printed guide which aids in precise placement of osteochondral graft material. Both of these materials can be fabricated using precise data obtained from radiological imaging.

### BRIEF SUMMARY OF THE INVENTION

The problems associated with conventional means of creating osteochondral and chondral grafts is addressed by the present invention by providing a reproducible process that may be used to fabricate size-specific, customized bio-printed musculoskeletal tissue in particular for use in various orthopedic applications.

The present invention also includes a custom, patient specific, 3-D printed guide for precise placement of osteochondral graft material that utilizes data obtained from radiologic imaging of the subject joint.

In one aspect of the invention, a method of repairing a defect on an articular surface of a bone within a joint is provided. The method includes acquiring a data set of a defect on an articular surface of a bone within a joint to be repaired by radiological imaging; evaluating the data set for the location of the defect; marking said defect with computer software; transferring said data with said marked defect to a 3-D printer and printing out a guide that demarcates said defect as a cut-out portion in said guide, said guide including a first guide reference element thereon; performing an osteochondral biopsy on an area of the damaged joint away from the defective area to obtain osteochondral cells; culturing the osteochondral biopsy cells to create a biogel; loading the biogel into a 3-D bioprinter to create an osteochondral tissue plug and hardening said osteochondral tissue plug to create an osteochondral AUTOgraft; inserting a second guide reference element into said biopsy site, said second guide reference element for matingly engaging said first guide reference element; placing said guide on the articular surface of said bone over said defect such that said cut-out portion is in alignment with said defect and said first and second guide reference elements are in mating relationship; sculpting the surface of said defect in said bone to a predetermined depth and in precise alignment with the contours of said cut-out portion of said guide; positioning said osteochondral AUTOgraft into said cut-out portion and press fitting said AUTOgraft into the sculpted surface of the bone; and surgically closing the joint.

In another aspect of the invention, a 3-D printed guide structured to aid in the placement of osteochondral graft material on bone is provided. The 3-D printed guide includes a base portion that precisely mimics the surface of a bone or musculoskeletal tissue to be repaired, the base portion including a cut-out therewithin that corresponds to a defect on the surface of the bone or musculoskeletal tissue to be repaired; a first guide reference element 3-D printed on said base portion and configured to matingly engage a second guide reference element positioned on a surface of the bone or musculoskeletal tissue to be repaired.

### BRIEF DESCRIPTION OF THE DRAWINGS

For a better understanding of the invention, and to show how the same may be carried into effect, reference will now be made, by way of example, to the accompanying drawings, in which:

FIG. 1A depicts an articular surface of femoral condyle showing an articular osteochondral defect to be repaired.

FIG. 1B depicts a femoral condyle guide to be used in conjunction with the articular defect depicted in FIG. 1A.

FIG. 2A depicts an exemplary surface of a tibial plateau showing the area of the defect to be repaired.

FIGS. 2B and 2C depict guides that may be used in conjunction with a tibial plateau central defect and tibial plateau marginal defect, respectively.

FIG. 3A depicts an articular surface of a trochlea showing the area of the defect to be repaired.

FIG. 3B depicts a guide that may be used in conjunction with the trochlea.

FIG. 4A depicts an articular surface of the patella showing the area of the defect to be repaired.

FIG. 4B depicts a guide that may be used in conjunction with the repair of the patella.

FIG. 5 depicts one aspect of the invention showing the articular surface to be repaired on a bone; a guide including a first reference guide element thereon and a cut-out area that replicates the size, shape, and thickness of the articular surface to be repaired; a second reference guide element inserted into the articular surface; and a bioprinted osteochondral or chondral graft to be placed in the articular surface.

FIG. 6A depicts a hole drilled at a biopsy site in the articular surface of a femur.

FIGS. 6B and 6C depicts a second guide reference element that is inserted into the hole of FIG. 6A.

FIG. 7 illustrates the guide that may be used in the repair of an articular surface of a femur, the guide including a first guide reference element configured for mating with the second guide reference element of FIGS. 6B and 6C.

FIG. 8 illustrates the first guide reference element being placed in mating relationship with the second guide reference element.

FIG. 9 depicts placement of the guide on the articular surface of the femur exposing the defect in the articular surface subject to repair.

### DETAILED DESCRIPTION OF THE INVENTION

Unless defined otherwise, all technical and scientific terms used herein have the same meaning as is commonly understood by one of skill in the art to which this invention belongs.

In one aspect the present invention relates to a reproducible process that may be used to fabricate size-specific, customized bio-printed musculoskeletal tissue for use in various human and veterinary orthopedic applications. Articular cartilage grafts and osteochondral grafts for implantation into any joint are described herein as exemplary. However, the present invention may also be utilized with bone grafts, labral grafts, meniscal grafts, spine disc grafts and ligament grafts among other tissues. The process of the present invention will now be described.

Acquisition of Data:

Precise data is needed for creation of orthopedic tissue. We propose the use of high resolution CT arthrography of the body part in question. Conventional CT, electron beam CT or CT arthrography, Ultrasound, MRI, MR Arthrography or other radiological methods may also be utilized. If CT arthrography is used, the CT data, for example, may be acquired using thin slice spiral CT (0.6 mm or thinner) and these slices may be “over sampled” using the lowest spiral CT pitch, usually 0.4 to 0.6 spiral pitch. This data set from the CT scan will create extremely small, isotropic voxels of precise data. The precise acquisition of data will allow accurate reconstruction of the orthopedic tissue, and is essential to achieve bio printing of living tissue. The precise data obtained is also essential for the 3D creation of a precise guide, used to aid in accurate placement of the newly created chondral or osteochondral material. The data set obtained from the CT images is evaluated for the location of the defects. The area of the defect is then marked using CAD or SLS software as described below. After marking the area of the defect, the data is sent to a 3-D printer and the guide in accordance with the invention is printed out with the precise area of the defect cut-out from the guide.

The AUTOgraft (disclosed in detail below) is bioprinted to the exact shape of the cut-out. Alternatively, if 3D bioprinting is not desired, osteochondral or chondral tissue can be cut into an exact shape using the CT data which defines the defect. If a polymeric covering is used instead of living tissue, this material can also be cut into the precise shape using the CT data which defines the defective area to be replaced. Surgery will be performed using the guide to remove the damaged bone. The bioprinted or possibly pre-surgically cut articular AUTOgraft will fit exactly into the cut-out on the guide and into the area of surgically removed bone.

Precision of Data in the XY Plane:

Because precise CT data is being utilized, the precise shape, size and contour of the osteochondral tissue for implantation in all planes may be obtained. In the XY plane, tissue can be fabricated to fit all sized cartilage defects. The defects to be addressed may range in size from a few millimeters in size to the entire surface of the joint. Conventional methods of repairing chondral defects use standard shaped osteochondral ALLOgrafts and AUTOgrafts for replacement, which are usually rounded, square or rectangular in shape and have a standard predefined thickness. It is known that osteochondral defects in patients occur in all sizes and shapes, and therefore, the ability to create customized osteochondral grafts for implantation of every shape and thickness will be a major improvement over conventional techniques.

Precision of Data in the Z Plane:

In the Z plane, osteochondral grafts that have identical contours to the native tissue may be created. Conventional osteochondral grafts have a standard predefined surface contour and usually have the appearance of a disc or thin sheet. Because the present invention prints osteochondral and chondral material utilizing acquired CT data, the surface contour of the native chondral surface may be precisely mimicked. This is especially important in the patella-femoral joint of the knee, which is known to have a very variable surface contour. The improved replication of the native anatomy will allow improved fit and longevity of the implants of the present invention versus conventional sheet type osteochondral implants.

Transfer of Data and Post Processing:

The data set will be downloaded to a high resolution 3D printer. An example of a 3D printer which may be modified and used is be the Projet 7000 HD (3D systems, Rock Hill, S.C.) or other 3D printers. The data transfer to the 3-D bioprinter may be achieved via download of data from a high definition CD disc or via streaming data via the Internet. This data set, because of its inherent high resolution will allow precise reconstruction of the tissue sampled. The data will be manipulated or post processed on CAD or SLS software used by several companies (for example Terra recon, Vital Images). The data manipulation is important to remove any portion of the tissue that does not need to be replicated and to remove any artifacts. Only the tissue to be replicated will be sent to the printer for bio printing. The same data created will also be sent to a 3D printing facility to create a precise template or guide which will act as a stencil to allow the surgeon to accurately address the diseased portion of the joint surface.

Creation of Cell Lines:

It is intended that using the patient's own body tissue for replication (AUTOgraft). For example, if osteochondral tissue is to be replicated, then the patient's osteochondral tissue needs to be acquired. A CT guided large core osteochondral biopsy will be performed. The biopsy may be obtained from tissue within the joint in question or from another joint. The osseous and cartilaginous tissue may then be cultured using a number of culture media presently used, the most common being hydrophilic gel. The cartilage and the bone cells may be cultured separately and grown into a large volume of living tissue. Progenitor or stem cells or other cells or additives may also be added to the newly created tissue, which may be obtained from synovial tissue or may require a separate bone marrow aspiration from the patient.

Creation of New Bio-Printed Tissue:

The newly created cell lines may be maintained in an aqueous state in methods known to those of skill in the art. This liquid “biogel” or “bioink” will be the material used to create the new tissue. The tissue will be loaded into a 3-D bio printer, possibly with bone gel and cartilage gel loaded separately. Using the acquired data, the tissue in question will be bio printed, possibly on a mold created to model the tissue in question on sheets of biopaper. The gel printed on to a template or mold may be “hardened” into a solid form. Exposure to UV light or compression is a possible mechanism to accomplish this task. This new product will likely be an osteochondral tissue plug identical in size in the XYZ planes, however, likely not identical in consistency to articular cartilage. A period of “tissue hardening” will likely be needed in order to mature the newly created osteochondral graft. Mechanical stress to the tissue similar to that expected to occur in normal native tissue may be used in this process of “tissue hardening.” or other methods. This will result in a final tissue that is a new, precise AUTOlogous, customized bio printed osteochondral AUTOgraft.

Use of the CT Data in Creation of 3-D Osteochondral Guide and Osteochondral or Chondral AUTOgraft:

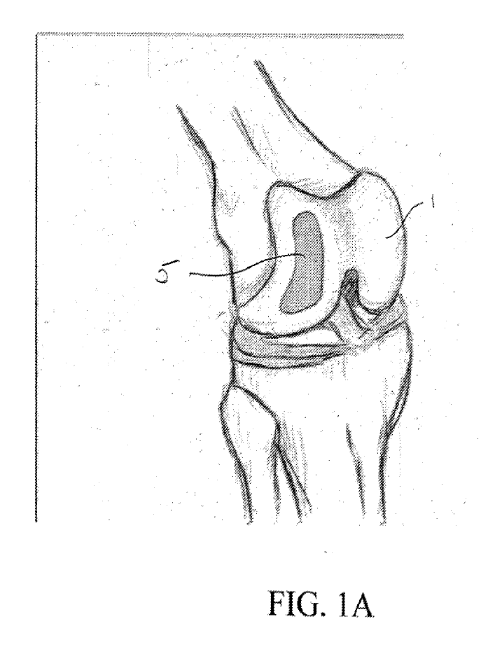

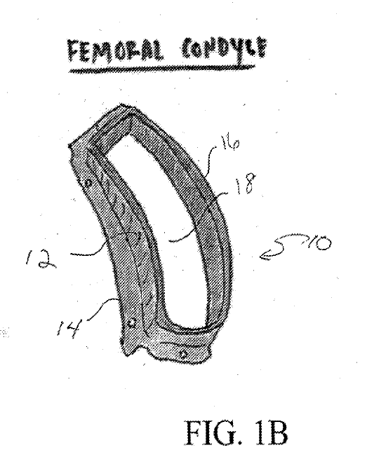

Using the data acquired from the CT scan, the area of tissue to be replaced will be identified, marked and manipulated on the computer generated images. Two processes will then occur. First, a sterilely created guide conforming to the exact shape of the articular surface of the joint will be 3D printed. The diseased area of the surface of the joint will be highlighted and a “cut out” within the guide using the CT data will be produced. Any material may be used to create the guide although it is preferable that the material be sturdy such as metal or heavy plastic by way of example. Second, an exact shaped bioprinted osteochondral graft corresponding exactly to the “cut out” in the guide and to the diseased joint surface will then be sterilely 3D bioprinted. Alternatively, using the CT data, an osteochondral or chondral graft can be cut into a precise shape pre-operatively. This osteochondral AUTOgraft will be the exact dimension and shape as the peripheral guide as depicted in the exemplary embodiments of FIGS. 1B, 2B, 3B, and 4B, 5, 6A-C, 7, 8 and 9.

The data used to create the 3-D printed guide may be obtained from the recipient joint surface using high-resolution radiologic imaging (including, but not limited to CT, CT arthrogram, electron beam CT or CT orthography, MR or MR arthrogram). The data may then be post processed using SLS software or other post processing software to identify the area of concern and optimize the 3-D printing of a guide. Using a 3-D printer, an intricate, detailed guide, unique to the patient's specific anatomy, is created. The 3-D printed guide can be made from any material. The guide has a unique 3-D shape which will fit over the recipient joint surface like a “hand in glove.” The recipient joint surface has unique contours, depressions, ridges and osteophytes and therefore the 3-D printed guide will fit uniquely on the joint surface. The guide is custom made to the patient's unique joint anatomy with differing shapes and contours from patient to patient.

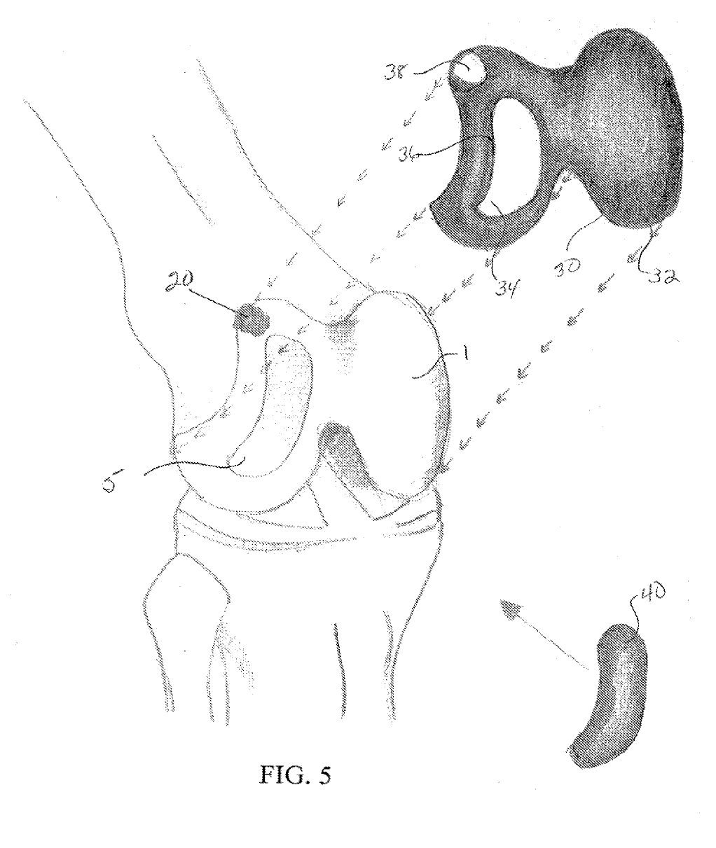

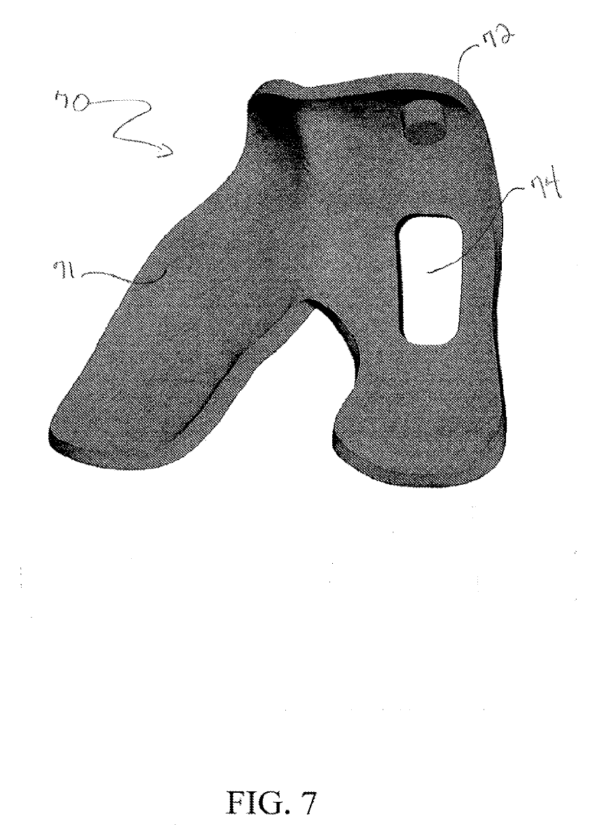

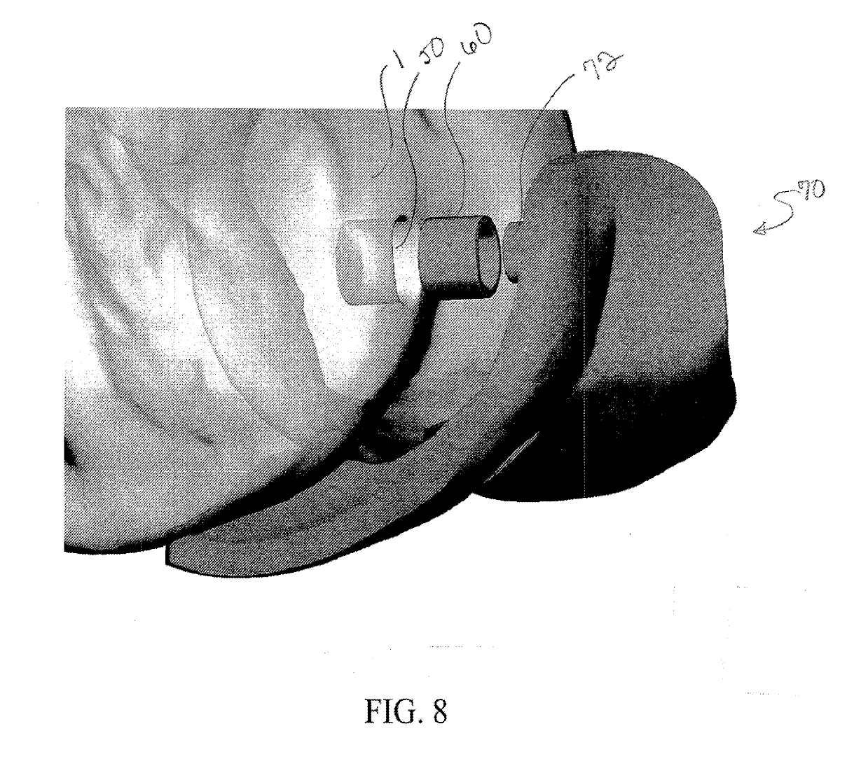

An additional safeguard to ensure precise placement of the guide will be 3-D printed into the guide. These additional safeguards are referred to herein as first and second guide reference elements. As best seen in FIG. 5, the first guide reference element may comprise a female portion while the second guide reference element may comprise a male portion. Alternatively, as best seen in FIGS. 7 and 8 the first guide reference element may comprise a male portion while the second guide reference element may comprise a female portion. The important aspect of the guide reference elements is that they be in mating relationship with each other to secure the guide to the articular surface of the bone during the surgical procedure to repair the defect.

Use of the 3-D Printed Guide:

Using standard surgical technique, the surface of the joint in question will be exposed. The guide will be placed on the recipient tissue surface as best illustrated in FIGS. 5, 8 and 9. The 3-D printed guide will have a “cut out” area in it, which corresponds to the portion of the surface of the joint which is damaged and needs to be to be replaced. The “cut out” portion will be determined from the previously obtained high resolution radiologic imaging. This “cutout” will guide the surgeon to the exact location of the damaged joint surface needing repair. The guide will act like a stencil, guiding the exact portion of the joint surface which needs to be replaced. Using the “cut out” as a guide, the surgeon may use a burr or other cutting device to debride the area of the joint surface bounded by the “cut out” area of the guide. Tissue will be removed from inside of the guide to a precise, predetermined depth and the depth of the tissue removed will be limited by a gauge on the cutting device which articulates with the 3D printed guide. A depth gauge will be placed on this tool to ensure uniform (accurate) depth of the defect created. The 3D printed guide may have elevated (thicker) or depressed (thinner) portions on its non-articular side. When the articulating portion of the cutting tool is pressed against the 3D printed guide, the areas of thicker guide will only allow shallower removal of diseased tissue, while the thinner guide areas will allow deeper removal of diseased tissue. Using this method, a precise depth or volume of diseased tissue can be precisely removed. The surgically created defect left after using the cutting device will mimic the exact shape of the “cut out” area of the 3-D printed guide and will be of a customized, predetermined depth.

Implantation of Newly Created Bio Tissue:

After the foregoing process of using the 3D printed guide has been accomplished, the osteochondral AUTOgraft will be introduced into the recipient and the osteochondral AUTOgraft will be placed in the surgically created defect. The osteochondral AUTOgraft will fit exactly in the defect in a “lock in key” manner, because the defect has been surgically created exactly to allow precise fit of the bioprinted osteochondral AUTOgraft. Moreover, the exact contour of the surface of the osteochondral graft will precisely follow the contour of the adjacent native tissue. This precise contour has been achieved by the precise bioprinting method described above using precise radiologic data. Alternatively, the osteochondral or chondral tissue can be cut using the radiological data, into a precise 3D shape in order to fit exactly into the surgically created defect. The implanted osteochondral AUTOgraft implanted will be held in place by the adjacent tissue in a “press fit” manner or may be fixed using bio-absorbable screws or pins or glue as has been described in the orthopedic literature or by any other appropriate means.

The joint may now be surgically closed in the standard fashion known to those of skill in the art.

The 3-D printed, customized guide may also be used with other available chondral replacement materials. For example, using high resolution radiological guidance including, but not limited to CT, CT arthrogram, electron beam CT or CT arthrography, MR or MR arthrogram, a 3D printed guide in accordance with the invention may be created to optimize precise placement of metallic, ceramic, porcelain and other types of orthopedic devices and prostheses in bones, joints and soft tissues. Those of skill in the art will recognize that the present invention may be used in, but not limited to, placement of total or partial joint surface replacement, bone replacement for bony defects, spine replacement and other types of musculoskeletal tissue replacement.

Referring now to the FIGS. the invention will be described. FIG. 1A depicts an articular surface 1 of a femoral condyle showing an articular osteochondral defect 5 to be repaired.

FIG. 1B depicts one aspect of a femoral condyle guide 10 in accordance with the invention to be used in conjunction with the articular defect 5 depicted in FIG. 1A. Guide 10 includes a base portion 12, flange 14 and edge 16. Edge 16 is configured to receive a sculpting tool or milling device known to those of skill in the art. Flange 14 may include a plurality of openings that can be used to secure the guide 10 to the articular surface of the bone during surgery. Guide 10 includes cut-out portion 18 which conforms precisely to the defect 5 being repaired.

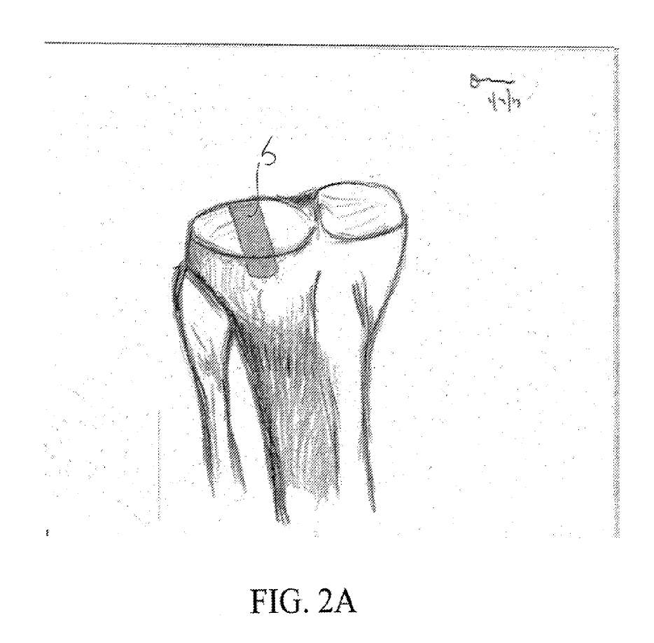

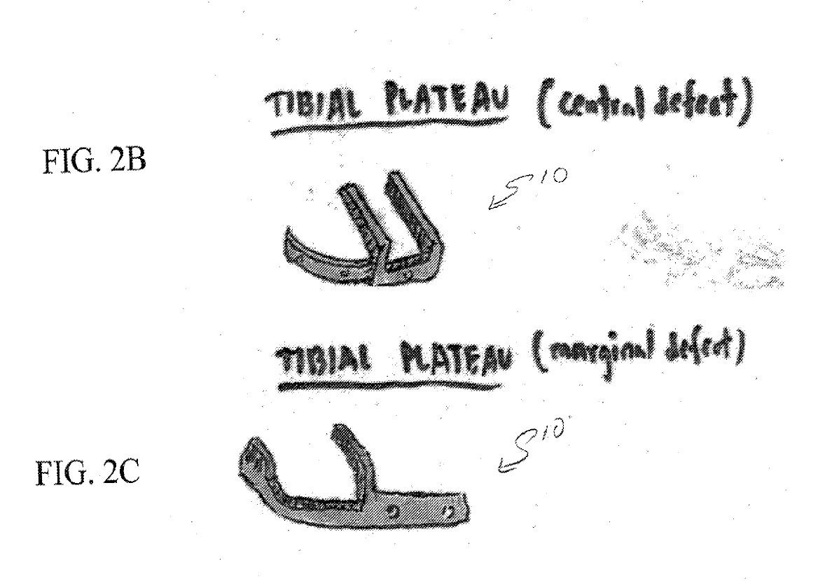

FIG. 2A depicts another surface of a tibial plateau showing the area of the defect 5 to be repaired. FIGS. 2B and 2C depict guides 10 that precisely conform to the articular surface of the bone to be repaired and may be used in conjunction with a tibial plateau central defect and tibial plateau marginal defect, respectively.

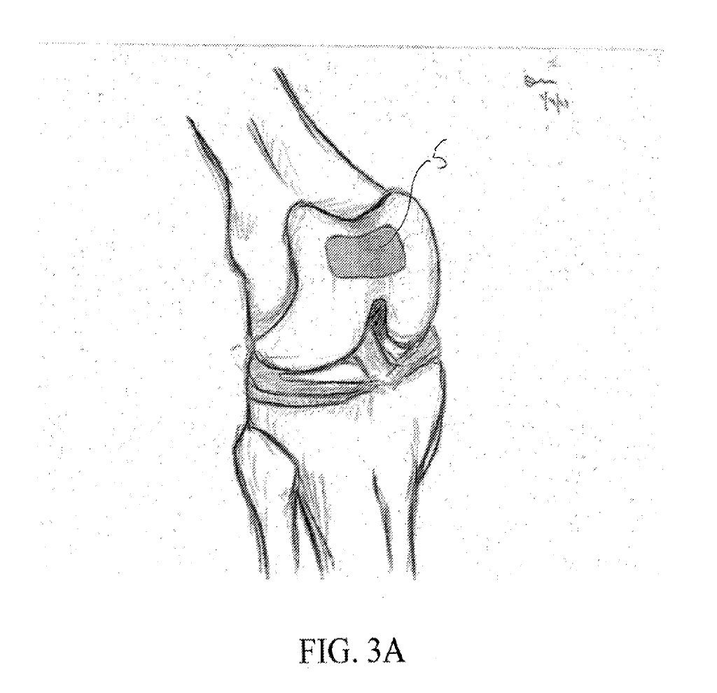

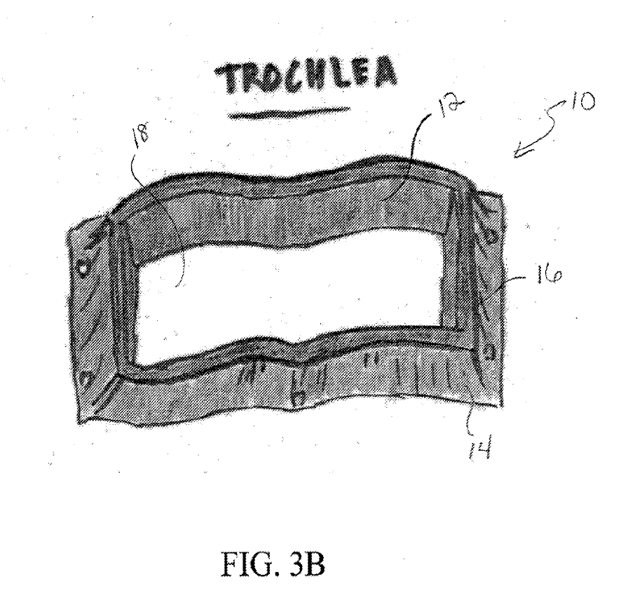

FIG. 3A depicts an articular surface of a trochlea showing the area of the defect 5 to be repaired. FIG. 3B depicts a 3-D printed guide 10 that conforms precisely to the articular surface of the trochlea being repaired in FIG. 3A and which includes a cut-out portion 18 that precisely conforms to the area of the defect.

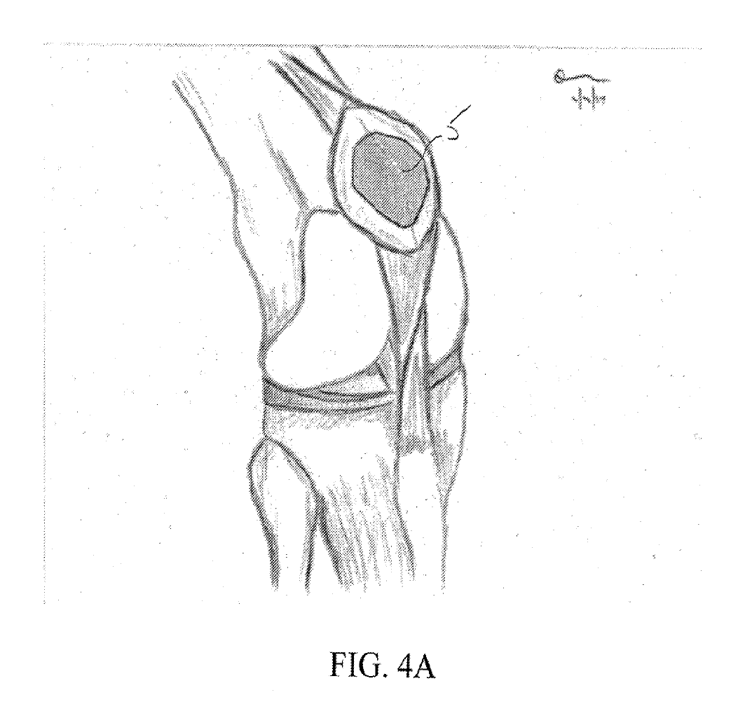

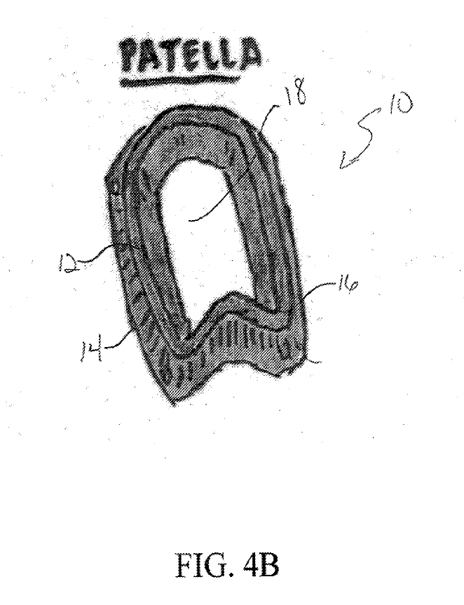

FIG. 4A depicts an articular surface of the patella showing the area of the defect 5 to be repaired FIG. 4B depicts a guide 10 that may be used in conjunction with the repair of the patella. The guide includes a base portion 12, flange 14 and edge 16. Edge 16 is configured to receive a sculpting tool or milling device known to those of skill in the art. Flange 14 may include a plurality of openings that can be used to secure the guide 10 to the articular surface of the bone during surgery. As with the guides depicted in FIGS. 1B, 2B-C and 3B guide 10 includes cut-out portion 18 which conforms precisely to the defect 5 being repaired.

FIG. 5 depicts another aspect of the invention and shows the defect 5 on the articular surface 1 to be repaired. A second guide reference element, male guide pin 20, has been inserted into the articular surface at the biopsy site and projects outwardly of the bone. Those of skill in the art will appreciate that the biopsy site is used by way of convenience but the guide pin 20 may be inserted into other parts of the articular surface. Guide 30 includes base portion 32 that is 3D printed from data acquired by radiologic imaging as previously discussed. Thus the base portion 32 conforms precisely to the articular surface of the bone to be repaired. In addition, cut-out portion 34 conforms precisely to the defect 5 to be repaired and precisely replicates the size, shape, and thickness of the articular defect 5. Guide 30 may optionally include an edge 36 that is configured to receive a sculpting tool such as a burr or milling device. Base portion 32 also includes first guide reference element, which is a female receiving guide pin opening 38 that matingly engages guide pin 20. 3-D bioprinted osteochondral or chondral graft, polymeric covering, or cut chondral graft material 40 precisely conforms to the cut-out portion 34 and to the defect 5 and in operation is press-fit into the defect, which has been sculpted or milled to remove the damage.

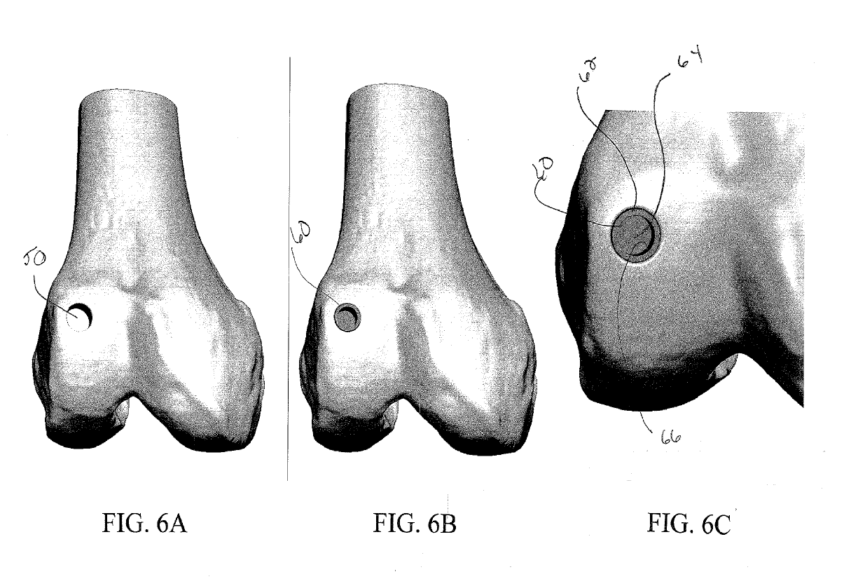

FIGS. 6, 7, 8 and 9 depict another aspect of the guide in accordance with the invention. FIG. 6A depicts hole 50 drilled at a biopsy site in the articular surface 1 of a femur. FIGS. 6B and 6C depict a second guide reference element, namely female guide plug receiving thimble 60 that is inserted into biopsy hole 50. Guide plug receiving thimble 60 includes a raised wall 62 surrounding floor 64. The outer circumference of raised wall 62 may be substantially round to allow for convenient placing it into hole 50. The inner circumference 66 of raised wall 62 may be in the shape of an arrowhead, arrow, square, round or may have any geometric shape such that the first guide reference element, male guide plug 72, as best seen in FIG. 8, matingly engages with it.

FIG. 7 illustrates the underside surface 71 of the 3-D bioprinted guide 70 that mates with the articular surface of a bone to be repaired. The guide 70 includes cut-out 74 which is configured to precisely replicate the defect in the bone. Guide 70 also includes first guide reference element, male guide plug 72, that is configured for mating with the second guide reference element, female guide plug receiving thimble 60.

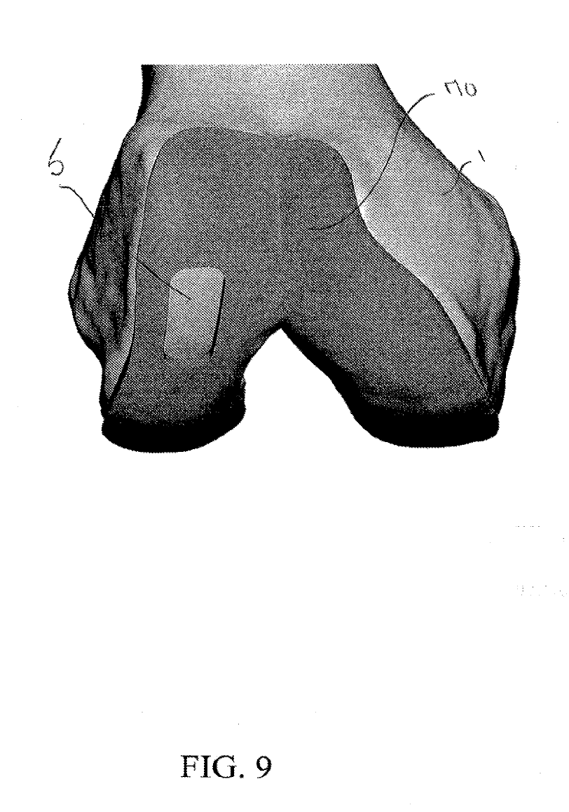

FIG. 8 illustrates the guide 70 being placed on the articular surface 1 of the bone to be repaired. Male guide plug 72 is shown being received in mating relationship with female guide plug receiving thimble 60. FIG. 9 depicts placement of the guide 70 on the articular surface of the femur exposing the defect and area to be repaired.

Those of skill in the art will appreciate that a depth guide structured to engage and articulate with the 3-D printed guide may be utilized with any of the embodiments disclosed herein.

Although the present invention has been described with reference to certain aspects and embodiments, those of ordinary skill in the art will appreciate that changes may be made in form and detail without departing from the spirit and scope of the invention.

What is claimed is:

1. A 3-D printed guide structured to aid in the placement of osteochondral or chondral graft material on bone comprising:
a base portion that conforms to the surface of a bone or musculoskeletal tissue to be repaired, said base portion including a cut-out therewithin, said cut-out structured to correspond to a surface of the bone or musculoskeletal tissue to be repaired;
a first guide reference 3-D printed on said base portion, said first guide reference configured to matingly engage a second guide reference positioned on the surface of the bone or musculoskeletal tissue to be repaired.

2. The 3-D printed guide of claim 1 further comprising an edge on said cut-out having a width configured to receive a bone milling tool.

3. The 3-D printed guide of claim 1 wherein said cut-out portion is configured from high-resolution radiologic imaging to precisely mimic a defect to be repaired.

4. The 3-D printed guide of claim 3 wherein said radiologic imaging is selected from CT imaging, CT arthrography, ultrasound, MRI, MR arthrography and combinations of the foregoing.

5. The 3-D printed guide of claim 1 further comprising a depth guide structured to engage and articulate with said 3-D printed guide.

6. The 3-D printed guide of claim 1 further comprising a guide plug in mating engagement with said receiving guide plug, said guide plug in engagement with the bone to be repaired.

7. The 3-D printed guide of claim 1 further comprising an implant received by said cut-out, said implant selected from biological, polymeric covering, ceramic, porcelain and metal implants.

8. The 3-D printed guide of claim 1 wherein said first guide reference comprises a male guide plug and said second guide reference comprises a female receiving guide thimble.

9. The 3-D printed guide of claim 1 wherein said first, guide reference comprises a female opening in said guide and said second guide reference comprises a male guide pin.

10. A method of repairing a defect on an articular surface of a bone within a joint comprising:
acquiring a data set of a defect on an articular surface of a bone within a joint to be repaired by radiological imaging;
evaluating the data set for the location of the defect;
marking said defect with computer software;
transferring said data with said marked defect to a 3-D printer and printing out a guide that demarcates said defect as a cut-out portion in said guide, said guide including a first guide reference;
performing an osteochondral biopsy on an area of the damaged joint not in the area of defect to obtain osteochondral cells;
culturing the osteochondral biopsy cells to create a biogel;
loading the biogel into a 3-D bioprinter to create an osteochondral tissue plug and hardening said osteochondral tissue plug to create an osteochondral AUTOgraft;
inserting a second guide reference into said biopsy site, said second guide reference for matingly engaging said first guide reference;
placing said guide on the articular surface of said bone over said defect such that said cut-out portion is in alignment with said defect and said first and second guide references are in mating relationship;
sculpting the surface of said defect in said bone to a predetermined depth and in precise alignment with the contours of said cut-out portion of said guide;
positioning said osteochondral AUTOgraft into said cut-out portion and press fitting said AUTOgraft into the sculpted surface of the bone; and
surgically closing the joint.

11. The method of claim 10 wherein said first guide reference comprises a male guide plug and said second guide reference comprises a female receiving guide thimble.

12. The method of claim 10 wherein said first guide reference comprises a female opening in said guide and said second guide reference comprises a male guide pin.
